(19) United States
(12) Patent Application Publication
(10) Pub. No.: US 2022/0380940 A1
(43) Pub. Date: Dec. 1, 2022

(54) VIOLACEIN-POLYMER COMPOSITE NANOFIBROUS MEMBRANE HAVING ANTIMICROBIAL EFFICACY AGAINST METHICILLIN-RESISTANT STAPHYLOCOCCUS AUREUS, AND MANUFACTURING METHOD THEREFOR

(71) Applicants: Korea Advanced Institute of Science and Technology, Daejeon (KR); CJ CheilJedang Corporation, Seoul (KR)

(72) Inventors: Il-Doo KIM, Daejeon (KR); Jiyoung LEE, Daejeon (KR); Jaewoo JANG, Seoul (KR); Changyub OH, Seoul (KR); Hyunae BAE, Seoul (KR); Jungmin LEE, Seoul (KR); Aeji JEON, Seoul (KR); Chungyup LEE, Seoul (KR)

(21) Appl. No.: 17/771,286

(22) PCT Filed: Oct. 23, 2020
(86) PCT No.: PCT/KR2020/014555

§ 371 Date: Apr. 22, 2022

(30) Foreign Application Priority Data
Oct. 24, 2019 (KR) 10-2019-0132945

(51) Int. Cl. D01F 1/10; D01D 5/00

(52) U.S. Cl. CPC D01F 1/103; D01D 5/0038; D10B 2401/13; D10B 2401/021

(57) ABSTRACT

Embodiments of the present disclosure relate to a violacein-polymer composite nanofibrous antimicrobial membrane and a method for manufacturing same, wherein the membrane comprises violacein having antimicrobial efficacy against methicillin-resistant Staphylococcus aureus (MRSA) caused by resistance to antibiotics and is formed such that one-dimensional nanofibers are three-dimensionally entangled, and can be used as an antimicrobial membrane for preventing and treating MRSA infections. Specifically, a solution in which violacein is uniformly mixed is prepared by dissolving a large amount of violacein in a solution with a polymer dissolved therein, and the solution is subjected to an electrospinning process to synthesize a nanofibrous membrane in which violacein is uniformly included inside/outside nanofibers without agglomeration. Thus, this is different from existing methods for applying a material to the surface of fibers.

### TECHNICAL FIELD

Embodiments of the present disclosure relate to a violacein-polymer composite nanofibrous membrane and a method for manufacturing the same, wherein the membrane contains a violacein having antibacterial efficacy against methicillin-resistant Staphylococcus aureus (MRSA). More particularly, these embodiments provide a nanofibrous membrane having antimicrobial efficacy against MRSA and a method for manufacturing the same, the nanofibrous membrane being manufactured by dissolving violacein with antimicrobial effects in a polymer solution and electrospinning the solution, and also provide a violacein-polymer composite nanofibrous antimicrobial membrane capable of preventing and treating MRSA infections and a method for manufacturing the same, which have long-lasting antimicrobial effects and allow firm adhesion to a bio skin for a long time, because violacein is uniformly dispersed inside and outside one-dimensional polymer fibers without agglomeration.

### BACKGROUND ART

MRSA is a type of Staphylococcus aureus (SA) bacteria that has developed resistance to antibiotics used to treat infections, which usually occurs in patients who have been in the hospital for a long time, or those who have had many medical procedures or surgeries, and which is often found in the course of treatment of wounds such as surgical sites. Recently, MRSA is found in local communities due to direct or indirect contact with out-patients and carriers, and can cause chronic otitis media, with a detection rate of 25 to 70% among people with chronic otitis media, which is raising awareness about the seriousness of MRSA.

The most common antibiotics currently used in South Korea to treat MRSA infections include Vancomycin, Teicoplanin, Linezolid, Quinolones, Rifampin, Trimethoprim-sulfamethoxazole (TMP-SMX), Clindamycin, Fusidic acid, Tetracyclines, and Tigecycline. Other antibiotics include Daptomycin, Telavancin, and Ceftaroline which have not been released in South Korea, and they are selectively used depending on the site and severity of infection.

Meanwhile, some of the most common methods used in hospitals and medical institutions to prevent and treat infections in wounds include a method of attaching a film or band to a wound to avoid exposure to external environments and a method of using dressings to apply antibiotics or other types of medicine. However, these methods do not eliminate the risk of secondary infections, requiring the patient to receive long-term and frequent treatment. Recently, an attempt has been made to attach a nanofibrous membrane containing an antimicrobial substance to a wound, with a study reporting that the growth of infecting bacteria was suppressed through this method. However, this method is problematic in that the nano membrane adheres weakly to skin or wounds, and in that the antimicrobial substance binds nonuniformly to the nanofiber since it agglomerates with increasing content, thus imposing a limitation to the content of the antimicrobial substance.

Notably, there is no vaccine for preventing MRSA, and washing hands with soap and water or an alcohol-based sanitizer is recommended for the prevention of MRSA. That is, individuals, local communities, and institutions such as hospitals can only take limited measures to prevent MRSA, and it is hard to treat infection sites locally in the case of infected people who have to undergo antibiotic therapy. Besides, the aforementioned common methods have limitations due to the risk of secondary infections, and therefore those infected with MRSA require a hospital stay. Moreover, owing to the large molecular size of conventional MRSA antibiotics, there are still challenges and difficulties in manufacturing antimicrobial substances in the form of membranes, such as agglomeration of the substances, low contents of the antimicrobial substances, and issues with adhesiveness to the skin. A recent finding showed that violacein, which is a blue-violet, bis-indole pigment having a low molecular amount, has antibiotic properties against MRSA, but it lacks in moisture and UV stability, making it less applicable to the conventional methods. In addition, conventional violacein coating methods have adhesion stability issues because violacein is coated onto the outer surface of fibers, and also has the problem of violacein getting detached from the surfaces of fibers.

In view of this, to overcome the above limitations, it is highly necessary to provide a technique of synthesizing and producing a structure which enables individuals and institutions to prevent or treat MRSA infections easily and effectively, can be used for a long time because it adheres firmly to skin and wounds, and allows a large amount of antimicrobial substance to be contained per unit volume without agglomeration.

### DISCLOSURE

### Technical Problem

The present disclosure provides a membrane and a method for manufacturing the same, wherein the membrane is capable of overcoming the inconvenience of conventional MRSA treatment requiring a hospital stay and the absence of preventive measures, and contains a large amount of violacein with antimicrobial properties against MRSA inside individual fibers constituting a three-dimensional network structure, and this membrane may be used for the prevention and treatment of MRSA infections by individuals and institutions, simply by attaching it to a bio skin.

The present disclosure provides a violacein-polymer composite nanofibrous membrane capable of preventing and treating MRSA infections, which can be mass-produced through a process of electrospinning a polymer composite solution containing violacein.

The present disclosure provides an antimicrobial membrane for preventing and treating MRSA infections and a method for manufacturing the same, wherein the membrane has high air permeability owing to the properties of nanofibers that adhere firmly to the skin and have a high specific surface area and high porosity, as opposed to conventional antimicrobial membranes.

### Technical Solution

An exemplary embodiment of the present disclosure provides a violacein-polymer composite antimicrobial nanofibrous membrane which includes a membrane composed of a plurality of nanofibers obtained by electrospinning a composite spinning solution that contains violacein and polymers, the membrane having antibacterial efficacy against methicillin-resistant Staphylococcus aureus (MRSA) because of the violacein.

According to an aspect, the violacein may be a powdered, blue-violet antimicrobial substance that is uniformly contained inside and on the surfaces of the nanofibers.

According to another aspect, the plurality of nanofibers may have one-dimensional structure, and the membrane composed of the plurality of nanofibers may have good water wettability despite the use of hydrophobic polymers in a matrix.

According to yet another aspect, the membrane may be composed of the plurality of one-dimensional nanofibers randomly entangled together or composed of a stack of a plurality of nanofibers aligned in a specific direction, wherein the thickness of the membrane is in the range of 5 μm to 100 μm, and the area of the membrane may be in the range of 1 cm² to 900 cm².

According to a further aspect, the diameter of each of the plurality of nanofibers may have a size distribution of 50 nm to 5 μm, and the nanofibers may include pores having an average diameter in the range of 10 nm to 25 μm, wherein the porosity is in the range of 40 to 90%.

According to a further aspect, the weight ratio of polymers in the membrane may be in the concentration range of 5 to 20% by weight of the total weight of the spinning solution, and the weight ratio of violacein in the membrane may be in the concentration range of 0.01 to 10% by weight relative to the total weight of the spinning solution.

Another exemplary embodiment of the present disclosure provides a method for manufacturing a violacein-polymer composite antimicrobial nanofibrous membrane, the method including: (a) preparing an electrospinning solution containing violacein, polymers, and a solvent; (b) synthesizing a membrane composed of a plurality of nanofibers by electrospinning the prepared electrospinning solution onto a substrate over a conductive current collector; and (c) separating the membrane composed of the plurality of nanofibers from the substrate.

According to one aspect, in the step (a), the polymers may include one or more types of polymer selected from the group consisting of poly-ε-(caprolactone) (PCL), chitosan, polyamide, poly-L-lactic acid, PLLA), poly(lactic-co-glycolic acid) (PLGA), polyanhydrides, polyacrylic acid, poly-N-isopropyl acrylamide, polyvinylidene fluoride (PVDF), poly(vinylidene fluoride-co-hexa fluoropropylene), perfluoropolymer, polyvinyl chloride (PVC), polyvinylidene chloride (PVDC), polyethyleneglycol dialkylether, polyethyleneglycol dialkylester, poly(oxymethylene-oligo-oxyethylene), polypropylene oxide (PPO), polyvinylacetate, poly(vinylpyrrolidone-vinylacetate)), polystyrene (PS), polyacrylonitrile (PAN), polymethylmethacrylate, polyamide, polyimide, poly(meta-phenylene isophthalamide), polysulfone, polyetherketone, polyetherimide, polyethylene terephthalate, polyethylene naphthalate, polyester, poly(1,1,2,2-tetrafluoroethylene), polyphosphazene, polyurethane, cellulose acetate, and copolymers and combinations thereof.

According to another aspect, in the step (a), the solvent may include a solvent selected from the group consisting of formic acid, acetic acid, phosphoric acid, sulfuric acid, m-cresol, tifluoroacetic anhydride/dichloromethane, water, N-methylmorpholine, N-oxide, chloroform, tetrahydrofurane, aliphatic ketones such as methyl isobutyl ketone and methyl ethyl ketone, aliphatic hydroxy compounds such as m-butyl alcohol, isobutyl alcohol, isopropyl alcohol, methyl alcohol, and ethanol, hexane, which is an aliphatic compound, tetrachloroethylene, acetone, glycols such as propylene glycol, diethylene glycol, and ethylene glycol, halogen compounds such as trichloroethylene and dichloromethane, aromatic compounds such as toluene and xylene, aliphatic ring compounds such as cyclohexanone and cyclohexane, esters such as n-butyl acetate and ethyl acetate, aliphatic ethers such as butyl cellosolve, acetic acid 2-ethoxyethanol and 2-ethoxyethanol, amides such as dimethylformamide and dimethylacetamide, and combinations thereof.

According to yet another aspect, in the step (a), the violacein may be obtained by isolating, extracting, and collecting violacein from cells or cell cultures produced from microorganisms grown to form violacein.

According to a further aspect, in the step (b), the diameter of the plurality of nanofibers and the size of pores between the plurality of nanofibers may be adjusted by applying a voltage of 1 to 30 kV through a high-voltage generator, adjusting the rotation speed of the conductive current collector at 50 rpm to 200 rpm, and adjusting the ejection rate of the solution in the range of 5 to 200 μl/minute.

According to a further aspect, in the step (b), the thickness and porosity of the membrane may be adjusted by adjusting the processing time of the electrospinning in the range of 10 minutes to 24 hours.

According to a further aspect, in the step (c), the plurality of nanofibers may be produced on the substrate, the nanofibers being solidified by natural evaporation of the solvent during electrospinning, and a membrane composed of the plurality of nanofibers may be separated as a freestanding membrane and therefore used alone without a support.

### Advantageous Effects

An antimicrobial nanofibrous membrane for preventing and treating MRSA infections according to the present disclosure may have good antimicrobial effects because it contains a large amount of violacein uniformly without agglomeration. Furthermore, the use of polymers with high water wettability provides good adhesion to skin or wounds and enables long-time use, and therefore allows for treating MRSA in a convenient way without a hospital stay. A membrane manufactured using an electrospinning technique capable of large-area synthesis may be used by individuals, local communities, and areas at risk of infection such as hospitals, and may effectively overcome the limitations on conventional methods of preventing and treating MRSA infections.

### DESCRIPTION OF DRAWINGS

The accompanying drawings, included as part of the detailed description in order to provide a thorough understanding of the present disclosure, provide embodiments of the present disclosure and together with the description, describe the technical features of the present disclosure.

FIG. 1 is a schematic diagram of an MRSA antimicrobial violacein-polymer composite nanofibrous membrane with violacein contained uniformly therein and nanofibers randomly arranged therein according to an embodiment of the present disclosure.

FIG. 2 is a block diagram of a method for manufacturing a violacein-polymer composite nanofibrous membrane using a process of electrospinning with a composite spinning solution prepared by mixing violacein and polymers according to an embodiment of the present disclosure.

FIG. 3 shows a chemical structure (a) and scanning electron micrograph (b) of violacein according to an embodiment of the present disclosure.

FIG. 4 is a view illustrating a principle of an electrospinning process for nanofibers in a random arrangement according to an embodiment of the present disclosure and a picture of actual equipment.

FIG. 5 shows an actual picture (a) and scanning electron micrograph (b) of a violacein-containing violacein-polymer composite nanofibrous antimicrobial membrane with one-dimensional nanofibers randomly arranged therein according to an embodiment of the present disclosure.

FIG. 6 shows an actual picture (a) and scanning electron micrograph (b) of a pure polymer nanofibrous membrane with one-dimensional nanofibers randomly arranged therein according to a comparative example of the present disclosure.

FIG. 7 shows pictures of the violacein-polymer composite nanofibrous antimicrobial membrane according to an embodiment of the present disclosure, when the contact angle between the membrane and water was measured to evaluate the contact angle properties.

FIG. 8 shows scanning electron micrographs of the membrane after washed with water (a) and the membrane after washed wish ethanol (b), when a stability test was conducted on the violacein-polymer composite nanofibrous antimicrobial membrane according to an embodiment of the present disclosure.

FIG. 9 shows a violacein-polymer composite nanofibrous antimicrobial membrane (a) and a pure polymer nanofibrous membrane, (b) with nanofibers randomly arranged therein, according to Embodiment 1 and Comparative Example 1 of the present disclosure, which were exposed directly to MRSA at ambient temperature when an MRSA antimicrobial test was conducted.

### BEST MODE

The present disclosure may be subjected to many changes and have several forms, and specific embodiments thereof are illustrated in the drawings and described in detail in the specification.

In describing the present invention, a detailed description of known technologies related to the present disclosure will be omitted when it is deemed that they may unnecessarily obscure the subject matter of the present disclosure.

Terms such as first, second, and the like may be used to describe various components, but these components should not be limited by the terms. The terms are used only for the purpose of distinguishing one component from another component.

Hereinafter, a violacein-polymer composite nanofibrous antimicrobial membrane having a three-dimensional structure in which one-dimensional nanofibers are randomly entangled, with MRSA antimicrobial violacein being uniformly contained inside and on the surfaces of the nanofibers, and a method for manufacturing the same, will be described in detail with reference to the accompanying drawings.

Embodiments of the present disclosure are characterized in that a violacein-polymer composite nanofibrous antimicrobial membrane composed of a plurality of nanofibers with violacein uniformly applied to the inside and surfaces thereof without agglomeration, wherein the membrane has a random nanofibrous network by uniformly dissolving violacein and polymers in a solvent capable of completely dissolving the violacein and the polymers and electrospinning the mixture while adjusting substrate rotation speed. The violacein-polymer composite membrane, manufactured by electrospinning a solution in which the violacein and the polymers are completely dissolved, contains a large amount of antibiotic substance per unit volume inside and on the surfaces of individual fibers, as distinct from and opposed to conventional methods of coating the surfaces of fibers with an antibiotic substance.

In conventional studies of antimicrobial membranes, attempts have been made to manufacture a membrane by electrospinning a spinning solution which is prepared by dispersing an antibiotic in a solution where polymer is melted. However, most antibiotics are hard to disperse uniformly in a polymer solution because of their large molecular size, thus leading to the problem of agglomeration of antibiotics over membrane fibers, and one limitation of the manufacture of such membranes is that they contain a low amount of antibiotics as the content of antibiotics is lowered to suppress agglomeration of antibiotics.

Besides, in a case where the membranes are made thick to increase antimicrobial effects, their air permeability is significantly lowered, making them unsuitable for use in skin and infection sites. Thus, it is essential to use a membrane having a large specific surface area and a porous structure to provide high air permeability and good antimicrobial effects. Hopefully, the synthesis of a membrane with pores of a constant size distributed therein, that allows for easy control of pore distribution, will lead to the development of an antimicrobial membrane with high reproducibility and good antimicrobial effects.

Moreover, conventional antimicrobial membranes are not suitable for attaching to the skin for a long time because of their low adhesiveness to skin and wounds, which requires the manufacture of a membrane that can be attached easily to human skin and remain attached for a long time. Especially, the use of a membrane with good water wettability will cause the membrane to adhere fully to the skin using moisture from the skin. Besides, it is necessary to manufacture an antimicrobial membrane that prevents infection with MRSA, because there is no currently available membrane with antimicrobial properties against MRSA, and the public and patients are given only a few options for the prevention and treatment of MRSA.

To overcome the aforementioned limitations and develop an antimicrobial membrane for preventing and treating MRSA that can be effectively used by both individuals and institutions, the present disclosure provides a method for synthesizing a violacein-polymer composite nanofibrous membrane with antimicrobial efficacy against MRSA by applying violacein having antimicrobial efficacy against MRSA uniformly onto polymer nanofibers aligned in a random orientation.

A nanofibrous membrane obtained by completely dissolving a powdered violacein in a solvent and electrospinning the mixture provides good antimicrobial properties compared to a pure polymer nanofibrous membrane containing no violacein. To manufacture an antimicrobial nanofibrous membrane having the above characteristics, a violacein-polymer composite nanofibrous antimicrobial membrane and a method for manufacturing the same are implemented through an efficient and easy-to-use process that allows for large-area synthesis.

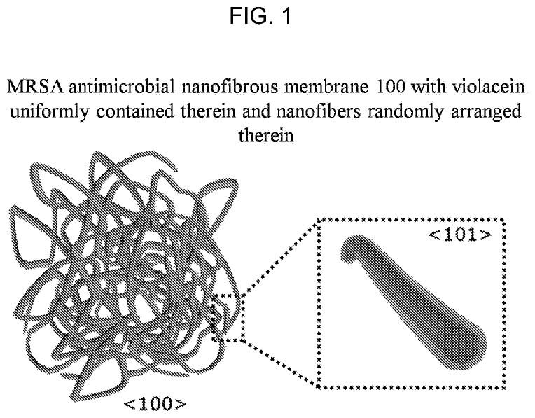

FIG. 1 is a schematic diagram of an antimicrobial nanofibrous membrane for preventing and treating MRSA, which is manufactured using randomly-arranged, violacein-polymer composite nanofibers 100 according to an embodiment of the present disclosure.

Embodiments of the present disclosure are characterized in that they provide randomly-arranged violacein-polymer nanofibers and violacein-polymer composite nanofibers aligned in a grid, which are attached easily to skin and wounds for the prevention and treatment of MRSA infections.

In this case, the diameter of the polymer nanofibers preferably ranges from 50 nm to 5 μm. The diameter of the nanofibers may be adjusted based on the viscosity and boiling point of a violacein-polymer composite solution, the amplitude of voltage applied to an electrospinning device, ejection rate, and nozzle radius. If the diameter of each nanofiber is 5 μm or greater, the porosity between the fibers may be significantly lowered, and the specific surface area may be decreased, thus impeding the flow of air between inside and outside. Also, the available area of contact between the violacein contained in the fibers and MRSA may be decreased, reducing the antimicrobial effects. That is, the use of a nanofibrous membrane with randomly-entangled nanofibers with a diameter range of 50 nm to 5 μm gives advantages in manufacturing an antimicrobial membrane having structural stability and high antimicrobial effects while maintaining high air permeability. Additionally, when manufacturing randomly-arranged nanofibers via electrospinning, the rotation speed and angle of a substrate over a conductive current collector, the ejection rate of a spinning solution, and the radius of a nozzle may be adjusted so as to adjust the intervals between the fibers and control the distribution of pore size in the range of 50 nm to 10 μm.

Moreover, the distribution state of the violacein contained in the polymer nanofibers is a very important factor. In order for the violacein to be contained uniformly in the nanofibers, the polymers and the violacein need to be completely dissolved in a solvent. To this end, the solvent may include a solvent selected from the group consisting of formic acid, acetic acid, phosphoric acid, sulfuric acid, m-cresol, tifluoroacetic anhydride/dichloromethane, water, N-methylmorpholine, N-oxide, chloroform, tetrahydrofurane, aliphatic ketones such as methyl isobutyl ketone and methyl ethyl ketone, aliphatic hydroxy compounds such as m-butyl alcohol, isobutyl alcohol, isopropyl alcohol, methyl alcohol, and ethanol, hexane, which is an aliphatic compound, tetrachloroethylene, acetone, glycols such as propylene glycol, diethylene glycol, and ethylene glycol, halogen compounds such as trichloroethylene and dichloromethane, aromatic compounds such as toluene and xylene, aliphatic ring compounds such as cyclohexanone and cyclohexane, esters such as n-butyl acetate and ethyl acetate, aliphatic ethers such as butyl cellosolve, acetic acid 2-ethoxyethanol and 2-ethoxyethanol, amides such as dimethylformamide and dimethylacetamide, and combinations thereof, and the violacein and the polymers need to be completely dissolved in the selected solvent so that the violacein is contained in the polymer solution without agglomeration. In this case, the solvent may be selected in consideration of the solubility of the violacein in the solvent to maximize the solubility of the violacein, thereby ensuring that a large amount of violacein is contained inside the polymer fibers.

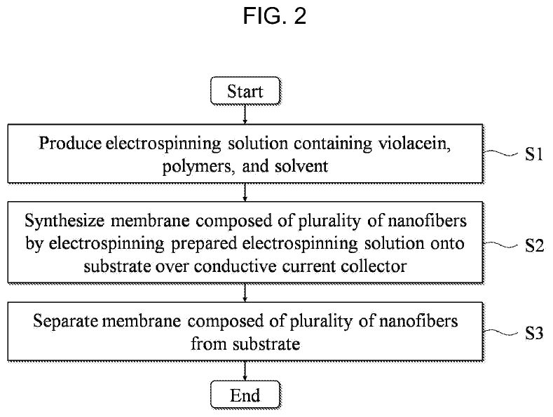

FIG. 2 shows a block diagram of a method for manufacturing a violacein-polymer composite antimicrobial nanofibrous membrane using a technique of electrospinning with a composite spinning solution prepared by mixing violacein and polymers, according to an embodiment of the present disclosure. As shown in the block diagram of FIG. 2, the method for manufacturing an MRSA antimicrobial violacein-polymer composite nanofibrous membrane may include a step S1 of preparing an electrospinning solution containing violacein, polymers, and a solvent; a step S2 of synthesizing a membrane composed of a plurality of nanofibers by electrospinning the prepared electrospinning solution onto a substrate over a conductive current collector; and a step S3 of separating the membrane composed of the plurality of nanofibers from the substrate. In the following, the above steps will be described in more detail.

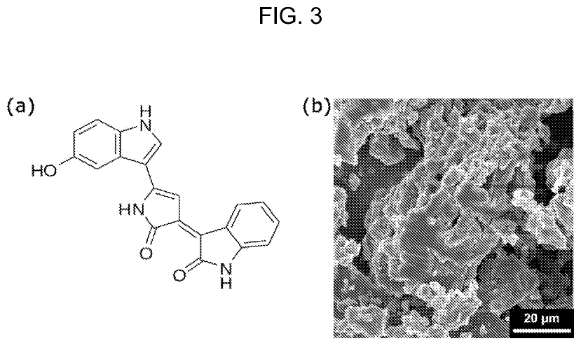

First, the step S1 of preparing an electrospinning solution containing violacein, polymers, and a solvent will be described. In the description of the violacein included in this step with reference to FIG. 3, the violacein has a bis-indol chemical structure as shown in (3) of FIG. 3, and (b) of FIG. 3 is a scanning electron micrograph of powdered violacein, in which case the powdered violacein is much less applicable because it agglomerates and has low solubility in an aqueous solvent. Accordingly, a method for manufacturing a membrane with violacein uniformly contained therein is essentially needed. Here, the violacein may be obtained by isolating, extracting, and collecting violacein from cells or cell cultures produced from microorganisms grown to form violacein. The polymers used in this step S1 are not limited to particular polymers as long as they are soluble in a solvent. Specifically, the polymers used herein may include at least one type of polymer, among polymethyl acrylate (PMA), polymethyl metaacrylate (PMMA), polyacrylic copolymer, polyvinyl acetate copolymer, polyvinyl acetate (PVAs), polyvinylpyrrolidone (PVP), polyvinyl alcohol (PVA), polyfurfuryl alcohol (PFA), polystyrene (PS), polystyrene copolymer, polyethylene oxide (PEO), polypropylene oxide (PPO), polyethylene oxide copolymer, polypropylene oxide copolymer, polycarbonate (PC), polyvinyl chloride (PVC), polycaprolactone, polyvinyl fluoride, polyvinylidene fluoride copolymer, polyimide, polyacrylonitrile (PAN), styrene acrylonitrile (SAN), polyvinyl alcohol (PVA), polycarbonate (PC), polyaniline (PANI), polyvinyl chloride (PVC), polyvinylidene fluoride), polyethylene terephthalate (PET), polypropylene (PP), and polyethylene (PE). In this case, the molecular mass of the polymers may be, but not limited to, 10,000 to 500,000, preferably 50,000 to 100,000 Da, more preferably 70,000 to 90,000 Da.

The selected polymers are dissolved in a commercial solvent such as N′-dimethylformamide, dimethylsulfoxide, N,N′-dimethylacetamide, N-methylpyrrolidone, distilled (DI) water, and ethanol. In this case, it is important to select a solvent that can maximize the solubility of the violacein, and the above solvents evaporate after electrospinning and are not therefore contained in the membrane.

In the manufacture of a spinning solution, a final electrospinning solution is produced by dissolving polymers in a selected solvent first and then adding violacein to the mixture. The solute-to-solvent mass ratio for forming the spinning solution preferably ranges from 1:16 to 1:13. Here, the mixture is stirred at ambient temperature. That is, the mixture is stirred for 3 to 24 hours at 50 to 200 rpm (preferably, 100 to 200 rpm) enough to make the polymers and the violacein completely dissolve in the solvent.

Next, the electrospinning solution produced in the step S2 is electrospun onto a substrate over a conductive current collector to synthesize a nanofibrous membrane. To this end, the electrospinning solution produced in the step S1 is transferred to a syringe with a proper capacity, and then a pressure is applied to the syringe at a constant speed using a syringe pump, so that a certain amount of solution is ejected at a certain intervals. The electrospinning system includes a high-voltage, grounded conductive substrate capable of rotation, a syringe, and a syringe nozzle. Here, the diameter of each of the plurality of nanofibers has a size distribution of 50 nm to 5 μm, and the nanofibers may include pores having an average diameter in the range of 10 nm to 25 μm since the intervals between the nanofibers are in the range of 10 nm to 25 μm. In this case, the porosity may be in the range of 40 to 90%.

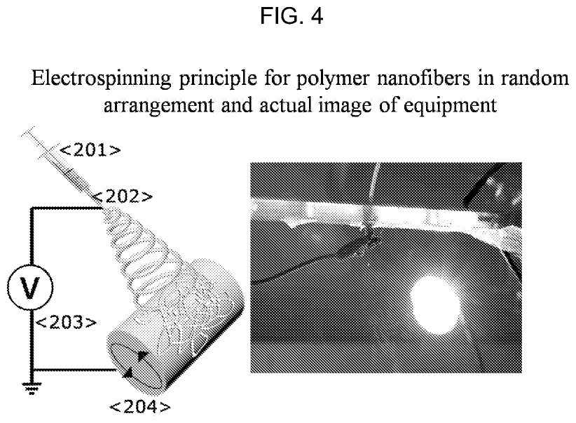

FIG. 4 is a view illustrating an electrospinning principle according to an embodiment of the present disclosure and an actual picture of devices constituting actual equipment. An electrospinning process will be described in detail with reference to FIG. 4. First, a nanofibrous membrane with nanofibers in a random arrangement is manufactured according to the electrospinning principle shown in FIG. 4. More specifically, a polymer solution containing a melted violacein is injected into a syringe 201, and a high voltage of 1 to 30 kV (preferably, 5 kV to 30 kV) is applied by a high-voltage application device (high-voltage generator) 204 between an injection needle 202 and a conductive current collector 203 to form an electric field. Then, the formed electric field causes a spinning solution ejected through an injection needle 202 to be extruded longitudinally in the form of one-dimensional nanofibers, thereby producing a membrane with nanofibers randomly arranged therein.

More specifically, the diameter of the plurality of nanofibers and the size of pores between the plurality of nanofibers may be adjusted by applying a voltage of 1 to 30 kV through the high-voltage application device 204, adjusting the rotation speed of the conductive current collector 203 at 50 rpm to 200 rpm, and adjusting the ejection rate of the solution in the range of 5 to 200 μl/minute. Also, the thickness and porosity of the membrane may be adjusted by adjusting the processing time of the electrospinning in the range of 10 minutes to 24 hours.

Finally, the step S3 is a process of separating a violacein-polymer composite nanofibrous membrane from a substrate, the membrane having a structure in which nanofibers functionalized with the violacein synthesized in the step S2 are randomly entangled together. The substrate and the nanofibrous membrane may be attached by an electrostatic or physical attractive force. In this case, the plurality of nanofibers may be produced on the substrate, the nanofibers being solidified by natural evaporation of the solvent during electrospinning, and a membrane composed of the plurality of nanofibers may be separated as a freestanding membrane and therefore used alone without a support.

Here, the violacein is a powdered, blue-violet antimicrobial substance, that may be contained uniformly inside and on the surfaces of the nanofibers. Each of the plurality of nanofibers constituting the membrane may have one-dimensional structure, and the membrane composed of the plurality of nanofibers has good water wettability despite the use of hydrophobic polymers in a matrix. Also, the membrane may be composed of the plurality of one-dimensional nanofibers randomly entangled together or composed of a stack of a plurality of nanofibers aligned in a specific direction. The thickness of the membrane may be in the range of 5 μm to 100 μm, and the area of the membrane may be in the range of 1 cm² to 900 cm². Also, the weight ratio of polymers in the membrane may be in the concentration range of 5 to 20% by weight of the total weight of the spinning solution, and the weight ratio of violacein in the membrane may be in the concentration range of 0.01 to 10% by weight relative to the total weight of the spinning solution.

The present disclosure is described in detail below through embodiments and comparative examples. The embodiments and comparative examples are intended to merely describe the present disclosure, and the present disclosure is not limited to the following examples. It would be obvious for a person skilled in the art that various changes and modifications are apparent within the scope of this description and the technical spirit and such changes and modifications definitely are included in the scope of the attached claims.

### Embodiment 1: MRSA Antimicrobial Violacein-Polymer Composite Nanofibrous Membrane Containing Violacein, with Nanofibers Randomly Arranged Therein

The following synthesis process will be conducted to synthesize a violacein-polymer composite antimicrobial nanofibrous membrane, with violacein contained inside and on the surface of each nanofiber without agglomeration.

First, a nanofibrous membrane is synthesized according to the electrospinning principle explained with reference to FIG. 4, with nanofibers randomly entangled together. Here, the diameter of the synthesized nanofibers preferably ranges from 50 nm to 5,000 nm to provide stable mechanical properties and achieve a membrane with a high specific surface area and high porosity.

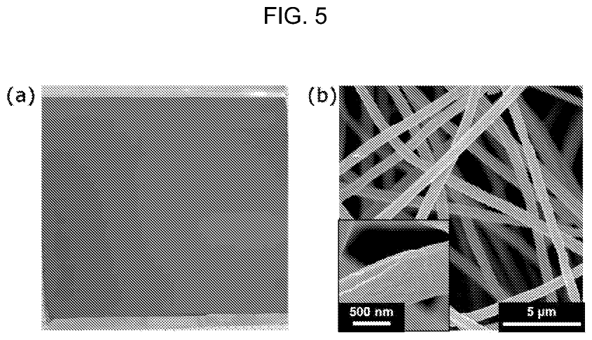

(a) and (b) of FIG. 5 show an actual picture and scanning electron micrograph of a violacein-polymer composite nanofibrous antimicrobial membrane containing violacein, with the nanofibers produced in the above process randomly arranged therein. The diameter of the nanofibers ranges from 50 nm to 5,000 nm, and the violacein is applied uniformly without agglomeration on the surfaces of the fibers because the surfaces of the fibers are smooth. Since the violacein is made into fibers by spinning, while uniformly dispersed in the polymer solution, the violacein is distributed uniformly over the inside and surfaces of the fibers. Moreover, the manufactured membrane may have a blue-violet color due to the violacein, and it was observed that the violacein was applied on the inside and outside of the nanofibers. The intervals between the fibers may be adjusted by controlling the voltage between a substrate over a conductive current collector and an injection nozzle and the rotation speed thereof, and the distribution of pores between the fibers may be freely adjusted in the range of 10 nm to 25 μm.

### Comparative Example 1: Pure Polymer Nanofibrous Membrane with Nanofibers Randomly Arranged Therein

Comparative Example 1 involves the synthesis of a pure polymer nanofibrous membrane containing no violacein, with nanofibers randomly arranged therein, as opposed to Embodiment 1.

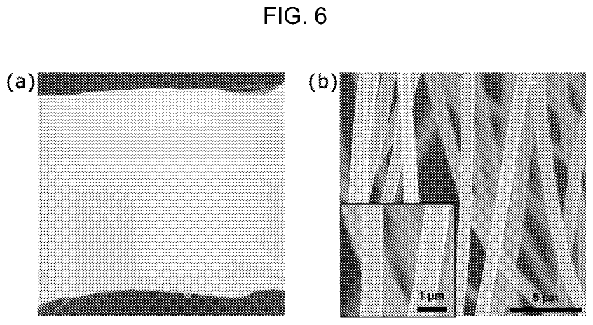

FIG. 6 shows an actual picture ((a) of FIG. 6) and scanning electron micrograph ((b) of FIG. 6) of polyacrynotrilie (PAN) nanofibrous membrane with nanofibers randomly arranged therein, formed by the typical electrospinning process shown in FIG. 4. In comparison to the violacein-containing nanofibers which were synthesized through Embodiment 1, Comparative Example 1 exhibited a smaller diameter, from which it can be found out that the membrane of Embodiment 1 contains violacein. As opposed to the membrane of Embodiment 1 having a blue-violet color, the membrane of Comparative Example 1 was white-colored, from which it can be found out that this membrane is made up only of pure polymer (PAN) nanofibers.

### Experimental Example 1: Evaluation of Contact Angle Properties of MRSA Antimicrobial Violacein-Polymer Composite Nanofibrous Membrane with Violacein-Containing Nanofibers Randomly Entangled Therein

An evaluation of the contact angle properties for water was performed in order to assess skin adhesion properties by measuring the water wettability of a nanofibrous membrane to which violacein is applied, with the nanofibers produced in Embodiment 1 randomly entangled therein. A static contact angle measurement method was used to measure the wettability of a solid surface by a liquid. The violacein-containing nanofibrous membrane was loaded onto a substrate, and then water droplets were placed one by one on the nanofibrous membrane using a syringe containing a distilled water solution.

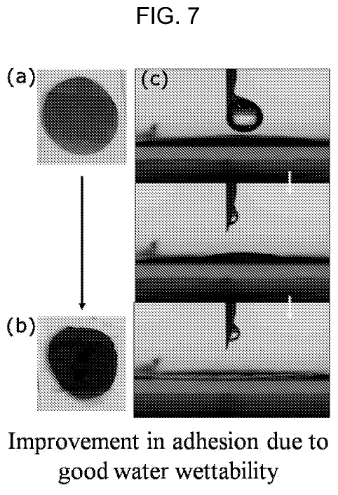

(a) of FIG. 7 shows an actual picture of a violacein-polymer composite nanofibrous membrane before testing, (b) of FIG. 7 shows an actual picture of the violacein-polymer composite nanofibrous membrane after testing, and (c) of FIG. 7 shows chronological pictures of a testing process in which one droplet of distilled water is placed on the membrane by a syringe containing a distilled water solution. As can be seen in (c) of FIG. 7, the droplet is quickly absorbed onto the membrane when it is placed on the violacein-polymer composite nanofibrous membrane, and the angle of the baseline and the angle of the droplet coincide and the membrane exhibited very high wettability for water. Also, the violacein-containing membrane has a deep indigo blue hue when exposed to the droplet, which is in contrast to the blue-violet color of the membrane of Embodiment 1. Particularly, in comparison to the powdered violacein having very low water solubility and wettability due to its hydrophobic nature, the violacein made in the form of a membrane has properties the existing powdered violacein does not have, thus making it usable for more applications. In addition, the good water wettability of this violacein may provide high adhesion between the skin and the membrane by supplying water to the skin or adding more water to the skin. Thus, the membrane used in Experimental Example 1 exhibited higher skin adhesion compared to the existing skin-attachable antimicrobial membrane.

### Experimental Example 2: Evaluation of Structural Stability of Violacein-Polymer Composite Nanofibrous Membrane with Violacein-Containing Nanofibers Randomly Entangled Therein

An evaluation of the structural stability of a violacein-containing nanofibrous membrane, with the nanofibers produced in Embodiment 1 randomly entangled therein, was performed by washing the membrane with distilled water and ethanol. The membrane manufactured in Embodiment 1 was immersed for several minutes in a beaker containing distilled water or ethanol and then dried, and structural changes to the membrane were observed before and after the immersion by scanning electron microscopic analysis.

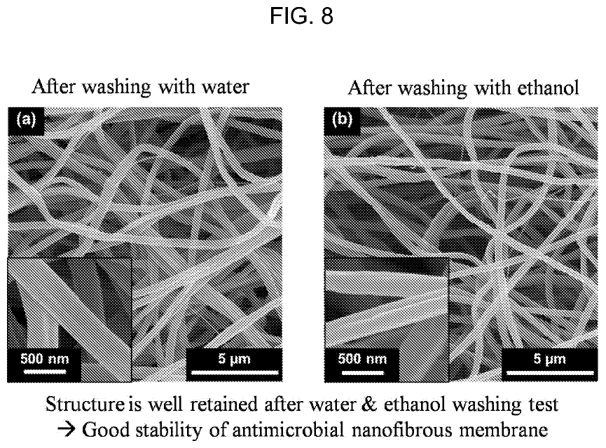

This will be described with reference to FIG. 8. (a) of FIG. 8 shows a scanning electron micrograph of the membrane after washed with water, and (b) of FIG. 8 shows a scanning electron micrograph of the membrane after washed wish ethanol. In comparison to the scanning electron micrographic image of Embodiment 1 before the immersion, both the scanning electron micrographic images after the immersion in (a) and (b) of FIG. 8 showed similar fibrous network structures in which the membrane remains stable after the washing. It was also found out that the membrane manufactured in Embodiment 1 forms a very stable nanofiber network and can be used for a long time.

### Experimental Example Related to Antimicrobial Testing: Evaluation of Antimicrobial Efficacy Against MRSA of MRSA Antimicrobial Violacein-Polymer Composite Nanofibrous Membrane with Nanofibers Randomly Arranged Therein and of Pure Polymer Nanofibrous Membrane Containing No Violacein

An evaluation of the antimicrobial properties against MRSA of a violacein-containing nanofibrous membrane, with the nanofibers produced in Embodiment 1 randomly entangled therein, was performed by exposing the membrane directly to MRSA. This experiment was conducted at ambient temperature, and the membrane manufactured in Embodiment 1 and the membrane manufactured in Comparative Example 1 were placed on a Petri dish where MRSA was grown, and then exposed for 18 to 24 hours, and the growth of MRSA around each membrane was observed.

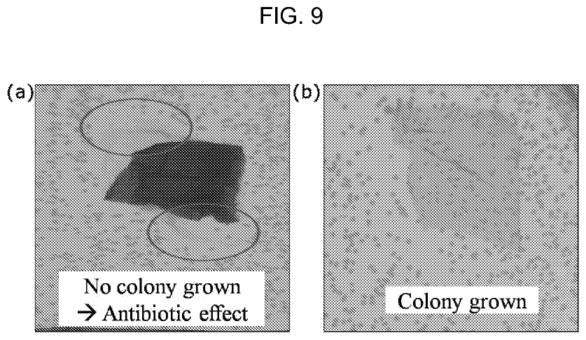

Referring to FIG. 9, FIG. 9 shows observations of colonies after exposure to MRSA, in which (a) of FIG. 9 is a digital image of a violacein-containing nanofibrous membrane manufactured in Embodiment 1, and (b) of FIG. 9 is a digital image of a pure polymer nanofibrous membrane containing no violacein manufactured in Comparative Example 1. It was found out that the membrane manufactured in Embodiment 1 significantly suppressed colony growth and exhibited good antibiotic (or antibacterial) efficacy, compared to Comparative Example 1. Notably, the spread of bacteria was better suppressed on the edge of the membrane than on other parts of the membrane. From this, it can be found out that the suppression of the spread and growth of bacteria starts from the edge of the membrane.

Such a nanofibrous membrane with antimicrobial properties against MRSA may be used for preventing MRSA infections in visitors and workers in places like hospitals subject to exposure to MRSA, and also may be used as a treatment membrane for preventing the growth of bacteria without a hospital stay by attaching it to wounds of infected patients. Moreover, the membrane may be attached to the skin for a long time owing to its good water wettability, and therefore may be used as an antimicrobial membrane for preventing and treating simple forms of MRSA that is economically efficient and gives convenience.

Embodiments of the present disclosure employ electrospinning and therefore allow for mass-producing a nanofibrous membrane capable of controlling the distribution of pores by easily adjusting the thickness of nanofibers and the intervals between them, which makes the membrane cost-effective and convenient-to-use. Also, an antimicrobial membrane manufactured by this method may be used for a long time by attaching it to skin and wounds owing to its good water wettability. By overcoming the limitations on the existing preventative measures against MRSA and treatment methods thereof, a violacein-polymer composite nanofibrous antimicrobial membrane may be conveniently used for preventing and treating MRSA by individuals or medical institutions such as hospitals.

### MODE FOR DISCLOSURE

The above description is merely illustrative of the technical idea of the present disclosure, and those skilled in the art to which the present disclosure pertains may make various modifications and changes without departing from the essential characteristics of the present disclosure. Therefore, the embodiments disclosed in the present disclosure are intended to describe the present disclosure, instead of limiting the technical idea of the present disclosure, and thus the scope of the technical idea of the present disclosure is not limited to these embodiments. The protection scope of the present disclosure should be interpreted by the following claims, and all technical ideas within the scope equivalent thereto should be construed as being included in the scope of the present disclosure.

1. A violacein-polymer composite antimicrobial nanofibrous membrane which comprises a membrane composed of a plurality of nanofibers obtained by electrospinning a composite spinning solution that contains violacein and polymers, the membrane having antibacterial efficacy against methicillin-resistant Staphylococcus aureus (MRSA) because of the violacein.

2. The violacein-polymer composite antimicrobial nanofibrous membrane of claim 1, wherein the violacein is a powdered, blue-violet antimicrobial substance that is uniformly contained inside and on the surfaces of the nanofibers.

3. The violacein-polymer composite antimicrobial nanofibrous membrane of claim 1, wherein the plurality of nanofibers has one-dimensional structure, and the membrane composed of the plurality of nanofibers has good water wettability despite the use of hydrophobic polymers in a matrix.

4. The violacein-polymer composite antimicrobial nanofibrous membrane of claim 1, wherein the membrane is composed of the plurality of one-dimensional nanofibers randomly entangled together or composed of a stack of a plurality of nanofibers aligned in a specific direction,
wherein the thickness of the membrane is in the range of 5 μm to 100 μm, and the area of the membrane is in the range of 1 cm² to 900 cm².

5. The violacein-polymer composite antimicrobial nanofibrous membrane of claim 1, wherein the diameter of each of the plurality of nanofibers has a size distribution of 50 nm to 5 μm, and the nanofibers include pores having an average diameter in the range of 10 nm to 25 μm,
wherein the porosity is in the range of 40 to 90%.

6. The violacein-polymer composite antimicrobial nanofibrous membrane of claim 1, wherein the weight ratio of polymers in the membrane is in the concentration range of 5 to 20% by weight of the total weight of the spinning solution, and the weight ratio of violacein in the membrane is in the concentration range of 0.01 to 10% by weight relative to the total weight of the spinning solution.

7. A method for manufacturing a violacein-polymer composite antimicrobial nanofibrous membrane, the method comprising:
(a) preparing an electrospinning solution containing violacein, polymers, and a solvent;
(b) synthesizing a membrane composed of a plurality of nanofibers by electrospinning the prepared electrospinning solution onto a substrate over a conductive current collector; and
(c) separating the membrane composed of the plurality of nanofibers from the substrate.

8. The method of claim 7, wherein, in the step (a), the polymers include one or more types of polymer selected from the group consisting of poly-ε-(caprolactone) (PCL), chitosan, polyamide, poly-L-lactic acid, PLLA), poly(lactic-co-glycolic acid) (PLGA), polyanhydrides, polyacrylic acid, poly-N-isopropyl acrylamide, polyvinylidene fluoride (PVDF), poly(vinylidene fluoride-co-hexa fluoropropylene), perfluoropolymer, polyvinyl chloride (PVC), polyvinylidene chloride (PVDC), polyethyleneglycol dialkylether, polyethyleneglycol dialkylester, poly(oxymethylene-oligo-oxyethylene), polypropylene oxide (PPO), polyvinylacetate, poly(vinylpyrrolidone-vinylacetate)), polystyrene (PS), polyacrylonitrile (PAN), polymethylmethacrylate, polyamide, polyimide, poly(meta-phenylene isophthalamide), polysulfone, polyetherketone, polyetherimide, polyethylene terephthalate, polyethylene naphthalate, polyester, poly(1,1,2,2-tetrafluoroethylene), polyphosphazene, polyurethane, cellulose acetate, and copolymers and combinations thereof.

9. The method of claim 7, wherein, in the step (a), the solvent includes a solvent selected from the group consisting of formic acid, acetic acid, phosphoric acid, sulfuric acid, m-cresol, tifluoroacetic anhydride/dichloromethane, water, N-methylmorpholine, N-oxide, chloroform, tetrahydrofurane, aliphatic ketones such as methyl isobutyl ketone and methyl ethyl ketone, aliphatic hydroxy compounds such as m-butyl alcohol, isobutyl alcohol, isopropyl alcohol, methyl alcohol, and ethanol, hexane, which is an aliphatic compound, tetrachloroethylene, acetone, glycols such as propylene glycol, diethylene glycol, and ethylene glycol, halogen compounds such as trichloroethylene and dichloromethane, aromatic compounds such as toluene and xylene, aliphatic ring compounds such as cyclohexanone and cyclohexane, esters such as n-butyl acetate and ethyl acetate, aliphatic ethers such as butyl cellosolve, acetic acid 2-ethoxyethanol and 2-ethoxyethanol, amides such as dimethylformamide and dimethylacetamide, and combinations thereof.

10. The method of claim 7, wherein, in the step (a), the violacein is obtained by isolating, extracting, and collecting violacein from cells or cell cultures produced from microorganisms grown to form violacein.

11. The method of claim 7, wherein, in the step (b), the diameter of the plurality of nanofibers and the size of pores between the plurality of nanofibers are adjusted by applying a voltage of 1 to 30 kV through a high-voltage generator, adjusting the rotation speed of the conductive current collector at 50 rpm to 200 rpm, and adjusting the ejection rate of the solution in the range of 5 to 200 μl/minute.

12. The method of claim 7, wherein, in the step (b), the thickness and porosity of the membrane are adjusted by adjusting the processing time of the electrospinning in the range of 10 minutes to 24 hours.

13. The method of claim 7, wherein, in the step (c), the plurality of nanofibers is produced on the substrate, the nanofibers being solidified by natural evaporation of the solvent during electrospinning, and a membrane composed of the plurality of nanofibers is separated as a freestanding membrane and therefore used alone without a support.
